(19) United States
(12) Patent Application Publication
(10) Pub. No.: US 2020/0316250 A1
(43) Pub. Date: Oct. 8, 2020

(54) BONE IMPLANT

(71) Applicant: Andrey PUGACH, Moscow (RU)

(72) Inventor: Andrey PUGACH, Moscow (RU)

(21) Appl. No.: 16/955,140

(22) PCT Filed: Dec. 20, 2018
(86) PCT No.: PCT/RU2018/050165

§ 371 Date: Jun. 18, 2020

(30) Foreign Application Priority Data
Dec. 20, 2017 (RU) 2017144895

(51) Int. Cl. A61L 27/02; A61K 6/831; A61L 27/04

(52) U.S. Cl. CPC A61L 27/025; A61K 6/831; A61L 27/042; A61L 2300/63; A61L 2430/38; A61L 2430/24; A61L 2430/12; A61L 2430/02

(57) ABSTRACT

The invention relates to the field of medicine and medical technology, namely to bone implants, and can be used in surgery, orthopedics, dentistry. Bone implants made of dense tough fibrous or fine-crystal silicate mineral aggregate or rock, jade, have high strength, resistance to crack formation, biological compatibility and the ability to integrate into bone tissue. The technical result—expanding the field of medical devices for implantation into bone tissue.

The invention relates to the field of medicine and medical technology, namely to bone implants. These are the implants that replace or supplement bone tissue or connect parts of the bones, typically they are fully or partially fixed in the bone tissue.

Implants of inorganic materials that are most commonly used as bone implants can be divided into two major groups—metallic implants and implants made of oxide materials, in particular aluminum oxide, zirconium oxide, silicon oxide.

Metallic implants are widely used after the discovery of osseointegration of titanium (Branemark, 1969). Pure titanium and its alloys (Ti with V, Al, Nb, Zr) are still used most frequently as a basis for implants. To improve biocompatibility, the surface of titanium implants is often modified. Sandblasting, etching and oxidation increase the microroughness and hydrophilicity of the surface of the titanium implant and lead to an increase in the speed of osseointegration.

The advantage of implants made of oxide-based materials is chemical resistance. Polycrystalline, in particular ceramic, as well as monocrystalline and vitreous oxide materials are used for the manufacture of implants. There is known an implant made of ceramics containing polycrystalline alumina (Hemke, 1975). Also known is a monocrystalline alumina implant (Hirabayashi, 1978). The compressive and tensile strength and the chemical resistance of the single-crystal alumina are very high.

Implants made from ZrO2-based oxide ceramics have high strength and high aesthetic quality; they are second in frequency of application after titanium implants. Yttrium-stabilized zirconia-based materials have high chemical and biological stability, high strength, relatively low Young's modulus (Yilmaz, 2007).

Silicon oxide based bone implants are known since 45S5 bioglass of the composition SiO2-P2O5-CaO—Na2O (Hench, 1971). Bioglass can be used in powder form as a filler for bone grafting in filling bone defects. Osseointegrated bioglass is later replaced with live bone tissue. It was originally assumed that osseointegration is induced by P2O5 and CaO from 45S5 bioglass that approximate the composition of the bioglass to the hydroxyapatite composition of the bone tissue. However it has been further found that both P2O5-free and CaO-free silicate glasses do also have osseoinductive properties (Hench, 1979).

Increased strength and reduced resorption of silicate materials is also known for artificial teeth made of lithium disilicate glass ceramics (Barrett, 1980) and apatite wollastonite glass ceramics (Yoshida, 1985). Silicate bioceramics of such compositions have excellent compatibility with the surrounding tissues and high strength, much higher than that of bioglass.

Biocompatibility and osseointegration ability of silica-containing implants, similar to titanium, depends on the surface structure. Experiments to determine the formation of hydroxyapatite crystals on various surfaces of silicon oxide materials were performed (Li, 1992) by placing samples in an SBF solution (SBF—simulated body fluid). SBF test is often used to evaluate the possibility of osseointegration and as an indicator of the biological compatibility of the materials. It has been found that the hydroxyapatite layer is formed in SBF on the surface of the silica gel with a developed rough surface, but this is not the case for silica glass with a smooth surface.

### SUMMARY OF INVENTION

In the present invention, the bone implant is completely or partially made of silicate rock or natural or artificial mineral aggregates with a dense tangled-fibrous or fine-crystalline structure known under the general name “jade”.

### Technical Problems

The most important properties of bone implants influencing the success of their application are mechanical strength, chemical resistance, biocompatibility, low electrochemical activity, Young's modulus, the ability to integrate into the bone (osseointegration).

Titanium implants have excellent strength, osseointegration ability, established medical protocols and long-term use. The disadvantage of metallic implants, including titanium implants is the electrochemical activity of metals that can cause different complications in the use of implants of titanium and its alloys (Pozhitkov, 2015).

Coating the surface of titanium implants with hydroxyapatite or bioglass may improve osteoplastic properties of the implant and reduce the electrochemical activity, but this effect is short-lived and poor due to resorption of the applied surface layer.

The alumina is very durable, but biologically inert, has a high Young's modulus and a positive zeta potential at physiological pH. This can lead to difficulties in osseointegration and bone resorption over time with prolonged use of the implant.

Possible ageing of zirconia ceramics in an aqueous environment makes an unreliable prognosis of the stability of zirconia implants during long-term use.

Silicate bioglass have high osseoinductive and osteoconductive properties, but have low strength, are fragile and susceptible to resorption with a long stay in the bone. These properties make it difficult to use them as a non-resorbable bone implants. Lithium disilicate and apatite-wollastonite silicate bioceramics have higher strength than that of biglasses, but have unknown prognosis of prolonged use.

These data show the promise of the use of bone implants from silicate materials in medicine, but the properties of the implants from many of such materials limit the scope of their application.

### Solution to Problem

In the present invention, to expand the range of medical devices for implantation into bone tissue and to obtain high-strength implants capable of osseointegration, bone implants are made from silicate mineral aggregates and rocks with dense tangled-fibrous or fine-crystalline structure, known under the general name “jade”.

As jade are known, among others, the following mineral aggregares and rocks: jadeite jade Na(Al,Fe)Si2O6, nephrite jade Ca2(Mg,Fe)5Si8O22(OH)2, xonotlite (xonotlite jade) Ca6(Si6O17(OH)2), vesuvianite (Californian jade) Ca10(Mg,Fe)2Al4((OH)4(SiO4)5(Si2O7)2), bowenite (new jade) (Mg,Fe)3(OH)4Si2O5, pectolite (Alaskan jade) NaCa2(Si3O8(OH)), hydrogrossular (Transvaal jade) Ca3Al2(SiO4)2(OH)4.

Like many minerals, mineral aggregates and rocks, jades may have slightly variable compositions depending on the formation conditions and deposit locations due to the phenomenon of isomorphism—the variability of the composition while maintaining the crystalline structure and material properties. Jades are massive and tough mineral aggregates and silicate rocks, occurring in nature, or synthetic materials of similar structure and composition. As it turned out unexpectedly, implants of these materials have the optimum characteristics for integration into the bone tissue.

These mineral aggregates and rocks are resistant to cracking, have dense tough structure, which provides high strength in combination with a relatively low modulus of elasticity. With the exception of jadeite jade, other jades are hydrosilicates. They are semi-transparent, translucent in thin layer and usually acquire a characteristic roughened matt surface. Since ancient times, jades were highly appreciated in the stone-cutting industry, a lot of tools (knives, axes) and art objects (cameos, statuettes, jewelry) were made of jade. The mechanical properties of these mineral aggregates can compete with titanium and steel, and compressive strength is superior to many alloys. However, the properties of jades related to the possibility of integration into the bone tissue, did not attract the attention of researchers.

The composition of jades includes silicon oxide, which makes them promising in terms of biological compatibility and ability to osseointegration. The fibrous structure of these silicates provides elasticity and microroughness required to form a strong bond with bone tissue. Jades as dielectrics are not involved in the electrochemical reactions, are hydrophilic, have a negative zeta potential at physiological conditions. Consideration of the set of jades properties allow us to hope for high quality of bone implants made of jades according to the present invention.

When evaluating the application possibilities of jades as a material for bone implants their high strength has been confirmed experimentally. Polished jade samples without cracks, inclusions and other defects were used in tests. It was confirmed that jades have a high compressive strength (more than 100 MPa), bending strength (more than 50 MPa), high impact strength, high fracture toughness (greater than 2 MPa/m½). Young's modulus of jades is relatively low and is comparable with the modulus of elasticity of titanium (100-250 GPa).

Test of the properties of materials in aqueous solutions showed that jades are hydrophilic with wetting angle being less than 90 degrees. The zeta potential is negative in jades, which is typical for materials capable of osseointegration.

These results show the promises of using jades for the production of bone implants. However, the interaction of these silicates with bone tissues has not been studied previously. The following results showed that implants made of jades have unexpectedly good biological compatibility and ability to osseointegration.

For initial evaluation of the biocompatibility jades were placed in SBF-solution according to the procedure (Li, 1992). After 1-4 weeks of the start of the experiment, hydroxyapatite crystals were formed on the surface of jades thus confirming the perspective of biological compatibility and the possibility of osseointegration of jade implants according to the invention.

To test in vitro the biocompatibility of jade implants, their interaction with mouse osteoblast cells was studied. It has been found that jade implants do not have a toxic effect on cells, osteoblasts attach and proliferate on the surface of jades.

Implantation of jade implants into the bone tissue was performed to verify the osseointegration. Cylindrical implants made of jades were placed in a hole in the tibia of guinea pigs. At the ends of the implants there were facets used to measure the torque. After 6 weeks of experiment the torque required for breakdown of implants was measured, and it was found that torque after engraftment of the implant exceeds the torque right after the inoculation of the implant into the bone tissue (more than 5 N/cm). These data confirm the ability to osseointegration of implants according to the present invention.

### Advantageous Effects of Invention

Bone implants made of jades according to the present invention have high mechanical properties, high hydrophilicity, high biological compatibility, ability to osseointegrate, low electrochemical activity, high aesthetic characteristics. An additional advantage of jade implants is their dielectric properties. Low conductivity reduces problems with MRI, CT and other medical procedures that use electrical signals. The properties inherent to jade implants show the promises of their use in surgery, orthopedics, dentistry.

### DESCRIPTION OF EMBODIMENTS

An exemplary and non-exhaustive list of jade implants according to the present invention comprises implants for replacement of bone tissue, volumetric bulk fillers in granular form for inclusion in the composition for filling bone tissue defects, bone screws, implants for bone fusion, fixing pins implants or pins for implants, implants for cranial reconstruction, orthopedic implants, transdermal osseointegrated implants, dental implants.

For orthopedic implants, the high strength of jade is of particular importance, which allows their use in implants in joints and heavy duty parts of endoprostheses. The group of orthopedic implants includes implants of hip joint, implants of knee joint, implants of shoulder joint, implants of elbow joint, implants of wrist joint, implants of ankle joint, implants of subtalar joint, implants of metatarsal joint, implants of joints of fingers, or in general, implants of joints, implants of spinal column, including implants of vertebra and implants of intervertebral disk, implants of head of radial bone, thumb implants, implants for osteotomy (high tibia osteotomy). Particularly preferred orthopedic implants are implants of joints, in particular, implant of hip joint and implant of knee joint. Implant of hip joint may be prosthesis of head and prosthesis of diaphysis (stem of endoprosthesis or diaphysis of femur) and cotyloid cavity prosthesis. The knee joint implant can include tibial and femoral components of the knee joint.

The next group of jade implants according to the present invention are transcutaneous osseointegrated implants (endoexoprostheses) which are integrated in the bone of the distal part of the joint on the one side, extra-bone part of the implant extends beyond the body through the tissues and the patient's skin, and the external part of the implant is used for fixation of external exoprotheses. For endoprostheses as well as for orthopedic implants the high strength of jade implants is highly valuable. A significant advantage for the endoexoprostheses made of jade can be the low electrochomic activity and the insulating properties of such implants. Group of percutaneous osteointegrated implants includes endoprothesis for legs, endoexoprostheses for hands, endoexoprostheses for fingers, endoexoprostheses for cosmetic surgery.

The group of dental implants includes the dental implant itself (spiral, cylindrical, conical or lamellar shape), dental abutment, dental implant with integrated abutment, dental crown, dental implant with integrated abutment and crown, dental beam, dental insert, dental pin.

Structure and color of implant body are extremely important for smile zone dental implants. It should be noted that the color of jade implants according to the invention may vary significantly depending on the composition of jade, its formation conditions, its source. Classic jade usually has green tones but it can be almost any color. White jades are also common, and is often valued more than green. Natural white jade, which has the color of “mutton fat”, is well known and has a high cost in some countries. It is widely used in the production of high-end jewelry. In nature there exist also white jadeite, xonothlite, vesuvianite, bowenite and pectolite jades. Synthetic jades can also be of any color, including white.

Jade dental implant may be helical, cylindrical, conical, or plate-like shape, demountable or combined with an abutment. In the detachable implant the anchoring of the dental abutment to the dental implant root is often carried out by means of an internal screw. The dielectric and mechanical properties of jade implant can provide electrochemical isolation of the internal fastening screw from biological fluids of the body. This insulation allows the use of screws of different materials, not only titanium, but also made from steel or other alloys for fastening parts of demountable dental implant. Other dental jade products such as dental crown, dental insert, dental pin can also get the benefits of osseointegration with the bone tissue of the tooth or jaw. Dental root implant, abutment and the crown can also be made of white jade to match to the patient's teeth tone. The consumer characteristics of white jade implants for the smile area, in particular cosmetic, exceed the consumer characteristics of metallic implants.

A preferred dental implant is an osseointegrated beam made from a greasum for attachment to the alveolar process. The shape and dimensions of the beam are selected under the patient, a platform is made on the alveolar process to contact the beam with the bone tissue, a recess corresponding to the shape of the lower part of the beam can be made. The beam, in which there are openings for fastening, can be fixed to the bone through the provided holes with the help of a prepared set of one or several screw endoosseous implants or bone screws. The resulting osteointegration leads to subperirest or endoosseous-subperiosteal implantation of combined beam complex and screw implant or bone screw. The combined implant is securely attached to the alveolar process and the bone plastic can be minimized. This implant is used as a base for fastening dental prostheses and uniformly distributes the chewing load to the area of contact of the beam with the bone. Application of such constructions is justified for the case of implantation in the molar area, or for the case of 3 or 4 bone tissue, or for the case of insufficient for endoosseous implantation of the volume of the alveolar process. A preferred dental implant is an osseointegratable jade beam properly adjusted for attachment to the alveolar bone. The shape and size of the beam may be chosen individually for the patient, the alveolar bone may be prepared for the area of contact of the bone tissue with the beam, shape adjustments of the bone may be made corresponding to the shape of the bottom of the beam. The beam framework, which have holes for fastening, can be fastened to the bone through the holes provided with the prepared set of one or more endosseous implants or bone screws. Osseointegration of the beam leads to complex subperiosteal or endosseous-subperiosteal implant combining integrated beam and helical implant or a bone screw. Thus a combined implant is securely fixed on the alveolar bone and bone grafting can be minimized. This implant may be further used as a base for fixing dental prostheses, chewing evenly distributes the load on the area of contact of beam with jaw bone. The use of such structures is justified for the case of implantation in the molar region, or in the case of bone type 3 or 4, or in the case of an insufficient volume of alveolar bone for endosseous implantation.

A particularly preferred dental implant is dental implant with integrated abutment which can be applied in one-step protocol implantation. Crown may be fixed immediately after implantation or after the successful healing of the implant. The use of an implant with an integrated abutment increases the convenience for the doctor and the reliability of the prosthesis for the patient. A further simplification of the implant design is a one-piece artificial tooth made of white jade, where the endosseous implant, abutment and dental crown are combined into a single medical device. Such an implant can be a standard size product, can be further adjusted locally or it can be made individually for a particular patient.

### EXAMPLES

### Example 1. Granules of Filler for Osteoplasty Manufactured from Pectolyte Jade, Also Known as Alaska Jade

The granules are sterilized, mixed with autologous bone chips, implanted as a filler into the bone tissue defect according to the agreed protocol.

### Example 2. The Intervertebral Disc Implant is made of Vesuvianite, Also Known as California Jade

It is manufactured according to the required shape and size, sterilized, implanted according to the agreed protocol.

### Example 3. Implant for Cranial Reconstruction Manufactured from Bowenite, Also Known as New Jade

It is manufactured according to required shape and size using CAD/CAM technology, sterilized, implanted according to the agreed protocol.

### Example 4: Endoprosthesis of Cotyloid Cavity of Hip Joint Manufactured from Nephrite Jade

It is manufactured according to the required shape and size, sterilized, implanted according to agreed protocol.

### Example 5. Integral Head and Stem of Hip Joint Endoprosthesis Manufactured from Jadeite Jade

It is manufactured according to the required shape and size, sterilized, implanted according to the agreed protocol.

### Example 6. Femoral Component of Knee Joint Endoprosthesis Manufactured from Xonothlite Jade

It is manufactured according to the required shape and size, sterilized, implanted according to the agreed protocol.

### Example 7. Helical Subgingival Endoosseous Dental Implant Manufactured from Hydroglossular, Also Known as Transvaal Jade

It is manufactured according to the required shape and size, sterilized, implanted according to agreed protocol.

Implant is used as an artificial tooth root for implantation into the alveolar process according to 2-step protocol.

### Example 8. Dental Screw Endoosseous Implant with an Integrated Abutment Manufactured from White Nephrite

It is manufactured according to the required shape and size, sterilized, implanted into the smile zone according to 1-step protocal.

### Example 9. Single-Block Artificial Tooth, in which the Endoosseous Screw Implant, Abutment, and Tooth Crown are Combined

It is manufactured according to the required shape and size from white nephrite, sterilized, implanted into the smile zone according to 1-step protocal.

The crown is adjusted in place.

### Example 10. An Osseointegratable Beam of White Xonothlite

It is manufactured according to the required shape and size using CAD/CAM technology.

The beam is fixed to the alveolar bone through holes in the beam using a xonothlite screw implant.

The beam is fixed to a groove in the bone tissue, which has been prepared according to the shape of the lower part of the beam.

The beam compensates for the lack of bone tissue in the smile zone, serves as the basis for dentures.

Bone implants made of jade, can be applied in the areas of medicine, like surgery, orthopedics, traumatology, stomatology.

### CITATION LIST

### Patent Literature

Heimke 1975 U.S. Pat. No. 3,919,723

Hirabayashi 1978 U.S. Pat. No. 4,122,605

Hench 1979 U.S. Pat. No. 4,171,544

Barrett 1980 U.S. Pat. No. 4,189,325

Yoshida 1985 U.S. Pat. No. 4,560,666

### Non Patent Literature

Branemark 1969 Scand. J. Plast. Reconstr. Surg., 3, 81-100

Hench 1971 J. Biomed. Mater. Res. Symposium No. 2 (Part 1), 117-141

Li 1992 J. Am. Ceram. Soc., 75 (S) 2094-2097

Yilmaz 2007 J. Prosth. Dent., 98, 120-128

Pozhitkov 2015 Plos One, 10 (10): e0140393

1. Bone implant, characterized in that the implant or at least a part of the implant is manufactured from jade—dense, tough, capable of osseointegration silicate mineral aggregate or rock with tangled-fibrous or fine-crystalline structure.

2. Bone implant of claim 1, wherein the jade is selected from the group including mineral aggregates and rocks: jadeite jade, nephrite jade, xonothlite jade, vezuvianite jade, pectolite jade, bowenite jade, hydrogrossular jade.

3. Bone implant of claim 1, wherein the implant is selected from the group including implants for replacement of bone tissue, volume fillers in granular form for inclusion in the composition for filling of bone tissue defects, wedge-shaped bone implants, bone screws, implants for bone fusion, fixing pins implant or fixing pins for implants, implants for cranial reconstruction, hip joint implants, knee joint implants, brachial joint implants, elbow joint implants, wrist joint implants, ankle joint implants, subtalar joint implants, metatarsal joint implants, fingers joints implants, or, as a whole, joints implants, spinal implants, implants of radial bone, thumb implants, implants for osteotomy.

4. Bone implant of claim 1, wherein the implant is selected from the group consisting of osseointegratable transcutaneous implants (endo exo prosthesis) including endo exo prosthesis of leg, endo exo prosthesis of hand, endo exo prosthesis of finger, endo exo prosthesis for cosmetic surgery.

5. Bone implant of claim 1, wherein the implant or part thereof is selected from the group consisting of dental articles: dental implants, dental abutments, dental implants with integrated abutment, dental crowns, dental implants with integrated abutment and crown, dental beams, dental inserts, dental pins.
